(19) United States
(12) Patent Application Publication
(10) Pub. No.: US 2008/0273572 A1
(43) Pub. Date: Nov. 6, 2008

(54) THERMAL DETECTOR FOR CHEMICAL OR BIOLOGICAL AGENTS

(75) Inventors: David J. Lawrence, Harrisonburg, VA (US); W. Gene Tucker, Churchville, VA (US); Thomas C. DeVore, Harrisonburg, VA (US); George L. Coffman, Dayton, VA (US); Ronald W. Raab, Harrisonburg, VA (US); Gerald R. Taylor, Harrisonburg, VA (US)

Correspondence Address: FOLEY AND LARDNER LLP;SUITE 500, 3000 K STREET NW, WASHINGTON, DC, 20007, US

(73) Assignee: James Madison University

(21) Appl. No.: 11/867,992

(22) Filed: Oct. 5, 2007

Related U.S. Application Data
(63) Continuation-in-part of application No. 11/809,716, filed on Jun. 1, 2007, now abandoned.
(60) Provisional application No. 60/810,682, filed on Jun. 2, 2006.

(51) Int. Cl. G01N 25/00; G01N 33/00; G08B 21/00

(52) U.S. Cl. 374/45; 340/584; 73/29.05

(57) ABSTRACT

A detector has a thermoelectric sensor and a reactive layer. The thermoelectric sensor is configured to sense heat and to provide an electrical signal based on the sensed heat. The reactive layer is coupled to the thermoelectric sensor and is reactive with an airborne chemical or airborne biological agent of interest. The reaction is exothermic or endothermic. When the airborne agent of interest reacts with the reactive layer, the reaction is detected by the thermoelectric sensor. The electrical signal provides an indication based on the reaction.

### CROSS-REFERENCE TO RELATED PATENT APPLICATIONS

This application is a continuation-in-part of U.S. application Ser. No. 11/809,716, filed Jun. 1, 2007, which claims the benefit under 35 U.S.C. § 119(e) of U.S. Provisional Patent Application No. 60/810,682 filed Jun. 2, 2006, the entire contents of which are hereby incorporated by reference in their entirety.

### STATEMENT REGARDING FEDERALLY SPONSORED RESEARCH OR DEVELOPMENT

This invention was made with government support from the National Institute of Standards and Technology under grant number 60NANB2D0108 (Critical Infrastructure Protection Program). In addition, this invention was made with government support from the National Science Foundation's Research Experience for Undergraduates (REU) Program, awards #DMR-0097449, 2001-2004 and #DMR-0353773, 2004-2007. The Government may have certain rights to this invention.

### BACKGROUND

The present application relates to detecting chemical or biological agents of interest, for example chemical or biological agents in the air or in liquids.

Technologies to detect airborne health threats inside buildings may be used to protect the safety of the occupants. Such technologies may be useful in a variety of industrial, environmental, and homeland security applications.

Devices are commercially available that give alerts for some common airborne pollutants. While these devices can provide early warning about changes in air quality, they cannot differentiate relatively common problems such as exhaust entering the building through the ventilation system from more serious health threats.

### SUMMARY

According to an exemplary embodiment, a detector comprises a thermoelectric sensor and a reactive layer. The thermoelectric sensor is configured to sense heat and to provide an electrical signal based on the sensed heat. The reactive layer is coupled to the thermoelectric sensor and is reactive with an airborne chemical or airborne biological agent of interest. The reaction is exothermic or endothermic. When the airborne agent of interest reacts with the reactive layer, the reaction is detected by the thermoelectric sensor which produces an electrical signal. The electrical signal provides an indication of the reaction.

According to another exemplary embodiment, a method comprises sensing heat on a material having an exothermic or endothermic reaction with an airborne chemical or airborne biological agent of interest. The method further comprises converting the sensed heat to an electrical signal indicative of the amount of heat sensed.

According to another exemplary embodiment, a thermoelectric alarm system comprises a plurality of detectors as described above. Each of the detectors comprises a reactive layer reactive with the same or different airborne chemical or airborne biological agent of interest. A control system is configured to receive the electrical signals, to determine when an alarm condition exists, and to provide an alarm output signal based on the determination.

### BRIEF DESCRIPTION OF THE DRAWINGS

FIGS. 1A-1B are top and cross-sectional views, respectively, of a thermoelectric sensor, according to an exemplary embodiment;

FIGS. 2A-2B are top and bottom views, respectively, of a thermoelectric sensor, according to an alternative embodiment;

FIG. 3 is a cross-sectional view of a thermoelectric sensor, according to another alternative embodiment;

FIG. 4 is a schematic diagram of a thermoelectric sensor, according to another exemplary embodiment;

FIG. 5 is a top view of an array of thermoelectric sensors, one of which has a reactive layer, according to an exemplary embodiment;

FIG. 6 is a graph of sensor output voltage from a thermoelectric sensor illustrating a hexane droplet test, according to an exemplary embodiment;

FIG. 7 is a perspective view of a flow tube apparatus used in some experiments, according to an exemplary embodiment;

FIG. 8 is a graph of sensor output voltage in response to injection of approximately 30 ppm ammonia into the flow tube test apparatus of FIG. 7, according to an exemplary embodiment;

FIG. 9 is a graph of integrated sensor output as a function of energy required to vaporize hexane droplets applied to a thermoelectric sensor, according to an exemplary embodiment;

FIG. 10 is a graph of sensor output over time of a copper oxalate-coated sensor to short-term exposures of approximately 0.060 ppm (injected into an inlet of a flowthrough test chamber at a concentration of 0.9 ppm) and approximately 1.4 ppm (injected into an inlet of the flowthrough test chamber at a concentration of 20 ppm) of ammonia vapor, according to an exemplary embodiment;

FIG. 11 is a graph of integrated sensor output as a function of the logarithm of the ammonia concentration, according to an exemplary embodiment;

FIG. 12 is a graph of changes in the infrared spectrum observed when ammonia reacts with copper oxalate, according to an exemplary embodiment;

FIG. 13 is a graph of sensor output voltage (amplified 100 times) in response to exposure of a sensor to a concentration of approximately 70 ppm ammonia using the flow tube test apparatus of FIG. 7, according to an exemplary embodiment;

FIG. 14 is a schematic diagram of a detector for biological agents of interest, according to an exemplary embodiment;

FIG. 15 is a schematic diagram of a detector for the anthrax spore protein BclA, according to an exemplary embodiment;

FIG. 16 is a schematic diagram of a detection and alarm system, according to an exemplary embodiment; and

FIG. 17 is a flowchart illustrating a method of detecting an airborne chemical or biological agent of interest, according to an exemplary embodiment.

### DETAILED DESCRIPTION

Described herein is a system and method for detecting airborne or liquid chemical or biological agents of interest or substances of concern. Also described is a system and method for detecting airborne or liquid chemical or biological agents of interest which is inexpensive to mass produce. Also described is a system that will detect several types of chemical and biological agents. Further described is a system and method to alert persons or operating systems of the presence of an agent of interest to, for example, allow them to initiate emergency procedures such as changing operation of the building ventilation system or advising occupants to take protective action (e.g., to don masks, go to a designated place in the building, exit the building, etc.).

The teachings herein extend to those embodiments which fall within the scope of the appended claims, regardless of whether they accomplish one or more of the above-mentioned objectives.

While the exemplary embodiments will be described with reference to the detection of airborne chemical and biological agents of interest, the teachings herein may be applied to detecting liquid agents of interest, whether chemical, biological, or otherwise.

In accordance with one embodiment, there is provided a detector, comprising (i) a thermoelectric sensor configured to sense heat and to provide an electrical signal based on the sensed heat and (ii) a reactive layer coupled to the thermoelectric sensor which is reactive with an airborne chemical or airborne biological agent of interest, the reaction being exothermic or endothermic. When the airborne agent of interest reacts with the reactive layer, the reaction is detected by the thermoelectric sensor and the electrical signal provides an indication based on the reaction. The thermoelectric sensor may be any thermoelectric sensor that can be configured to sense heat and to provide an electrical signal based on the sensed heat. The reactive layer may be any reactive layer that is reactive in an exothermic or endothermic reaction with an airborne chemical or airborne biological agent of interest. The airborne chemical or airborne biological agent of interest may be any such agent of interest, including but not limited to agents that pose a threat to animal, human or public health or safety. Methods using such detectors to detect agents of interest also are provided.

In accordance with another embodiment, there is provided a method comprising sensing heat on a material having an exothermic or endothermic reaction with an airborne chemical or airborne biological agent of interest. In some embodiments, the method further comprises converting the sensed heat to an electrical signal indicative of the amount of heat sensed.

In accordance with another embodiment, there is provided a thermoelectric alarm system comprising a plurality of detectors as described above. Each of the detectors comprises a reactive layer reactive with the same or different airborne chemical or airborne biological agent of interest. The system may further comprise a control system configured to receive the electrical signals, to determine when an alarm condition exists (e.g., when at least a threshold amount of an agent of interest is detected), and/or to provide an alarm output signal based on the determination. Suitable control systems for carrying out one or more of these functions in response to an electrical signal are known and can be designed and implemented by the skilled practitioner.

Specific embodiments are described with reference to the figures. The skilled practitioner will understand that these embodiments are exemplary only, and do not limit the scope of the claims.

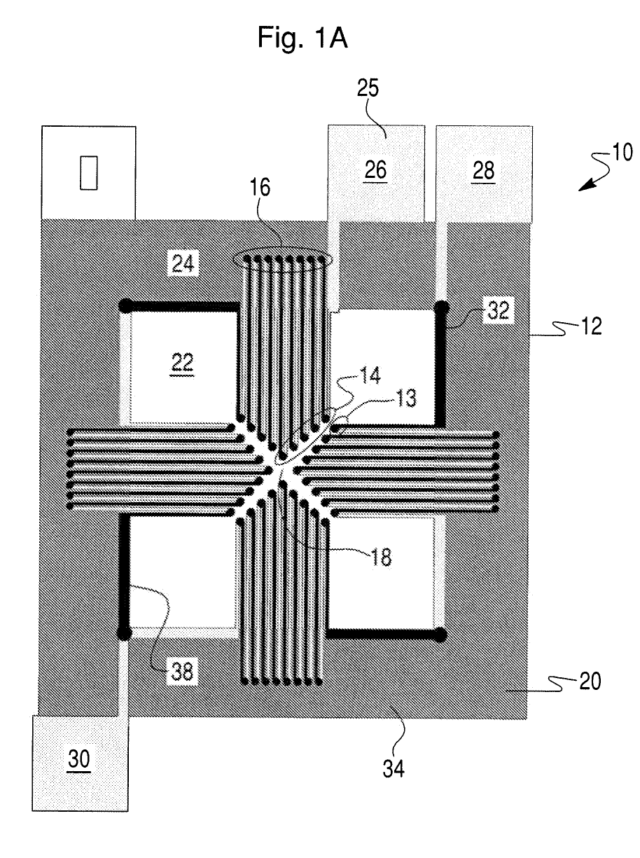

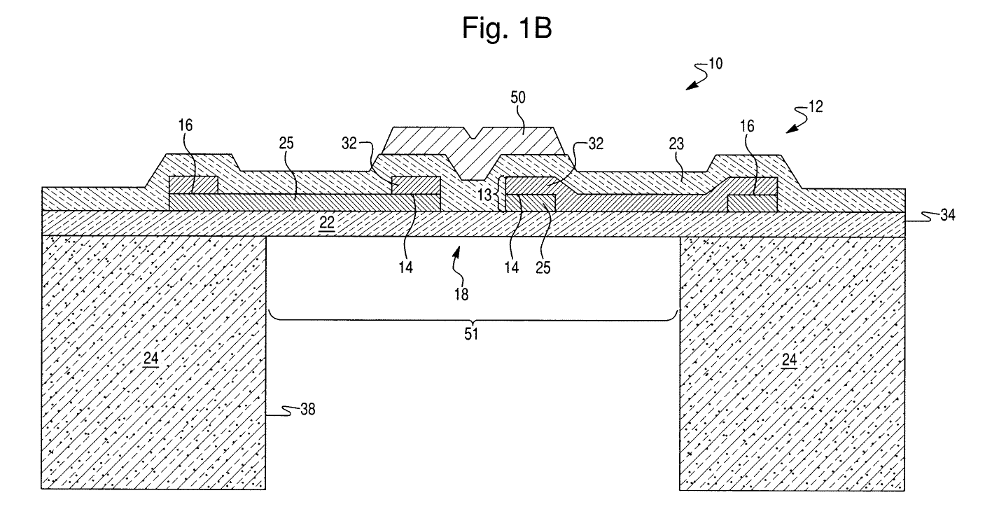

Referring first to FIGS. 1A and 1B, a detector 10 is shown in a top view and cross-sectional view, respectively, except that FIG. 1B shows detector 10 with a layer of reactive material 50, which is not shown in FIG. 1A. Detector 10 comprises a thermoelectric sensor 12 configured to sense heat and to provide an electrical signal based on the sensed heat. Sensor 12 may serve as a platform to detect temperature or heat differences. Sensor 12 may comprise a thermocouple 13 and may further comprise a thermopile. A thermopile comprises a plurality of series-connected thermocouples and provides a larger output voltage, and therefore a higher sensitivity, than a single thermocouple junction. Sensor 12 may alternatively comprise other thermoelectric sensing components, such as bolometers, calorimeters, etc.

Sensor 12 may have a sensitivity greater than about 4 V-s/J (Volt-seconds per Joule), or between about 1 V-s/J and 100 V-s/J. Sensor 12 may have a sensitivity to detect small temperature changes, such as temperature changes at or below about 1×10⁻³ degrees Celsius. Sensor 12 may be configured with sufficient sensitivity to distinguish air temperature changes from temperature changes created by substance reactions with a reactive layer, as will be described below. The sensitivity of sensor 12 may be increased, for example, by increasing the number of thermopile junctions to increase the output voltage. Additionally or alternatively, the sensitivity may be increased by increasing the thermal isolation between the sensing junctions and the reference junctions, for example, by thinning a membrane between a center and a perimeter of the device (e.g., via plasma etching). Thermal modeling may be used to determine the benefits of changes in geometry of materials of the sensor design, or of the geometry of the coatings, for detecting both gaseous and particulate substances, and suitable geometries may be selected for optimal detection of the agents of interest.

Sensor 12 comprises one or more sensing junctions 14 and one or more (typically an equal number of) reference junctions 16. In the illustrated embodiment, thirty-six sensing junctions 14 are disposed in a cluster near a center 18 of sensor 12 and thirty-six reference junctions 16 are disposed on or near an outer perimeter 20 of sensor 12. Alternatively, any number of thermocouples may be used, such as twenty-four, or preferably between about 1 and about 200 thermocouples, each having a sensing junction and a reference junction. In the illustrated embodiment, each thermocouple comprises an antimony conductor coupled to a bismuth conductor, though other conductive or semiconductive materials may be used, such as, nickel, chromium, aluminum, alloys, or doped silicon. Sensor 12 may comprise a membrane 22 (e.g., polyimide, polyethylene terephthalate, or other material, which may be a “drumhead” membrane) supported by a plate 24 (e.g., aluminum, a semiconductor, or other material), the membrane 22 supporting a plurality of thermocouples comprising reference junctions thermally grounded to plate 24.

Membrane 22 is shown in white in FIG. 1A. A first conductive material 25 is shown in light gray and includes signal output terminals 26, 28 and 30. Terminal 30 is configured to provide a signal representing heat sensed on half of sensor 12, the half selected based on which of terminals 26 and 28 are used as the reference to terminal 30. A second conductive material 32 is shown in black and comprises a material having a different voltage response to heat than first conductive material 25 to create the thermoelectric effect. Plate 24 is shown in dark gray and supports or is coupled to membrane 22. Plate 24 also may comprise a thermally conductive material, such as a metal, to maintain a thermal ground for reference junctions 16.

Sensor 12 may be a variety of shapes and sizes, including square, rectangle, circle, oval, and other shapes. Junctions 14 and 16 may be laid out in a variety of configurations. In the illustrated embodiment, thermocouples 13 are in a “+” or “T” configuration, with reference junctions 16 arranged linearly and sensing junctions 14 arranged in a “V” shape, with each “V” shape from four different sets of thermocouples meeting at center 18. Outer perimeter 20 may have an outside edge 34 sized 9 mm by 9 mm, though other sizes are contemplated, such as between about 0.1 mm by 0.1 mm and 20 mm by 20 mm.

Sensor 12 may be fabricated using any suitable techniques. For example, sensor 12 may be microfabricated using techniques for depositing and patterning materials 25 and 32 on membrane 22 and/or plate 24. The techniques may comprise one or more of thin film deposition, photolithography, etc., to form the thermocouple junctions between materials 25 and 32. Thermocouples 13 may be protected with a protective layer 23, such as a spin-coated polystyrene film, silicon dioxide, or other material, disposed or between a reactive layer 50 and thermocouples 13, or coating thermocouples 13. Layer 50 may be applied to protective layer 23. Layer 50 may be applied to thermocouples 13 either with or without an intervening protective layer 23.

In the embodiment of FIGS. 1A and 1B, sensor 12 may be fabricated on plate 24 having an aperture 38 smaller than perimeter 20, wherein sensing junctions 14 are disposed on membrane 22 (over aperture 38) and reference junctions 16 are disposed over plate 24. With this configuration, reference junctions 16 are “thermally grounded” in the sense that they are less responsive to rapid changes in ambient temperature. Aperture 38 and membrane 22 define a drumhead area 51.

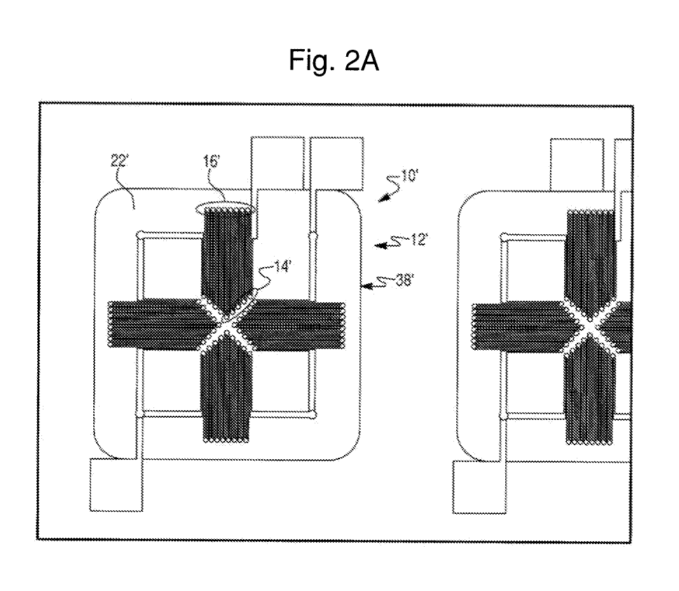

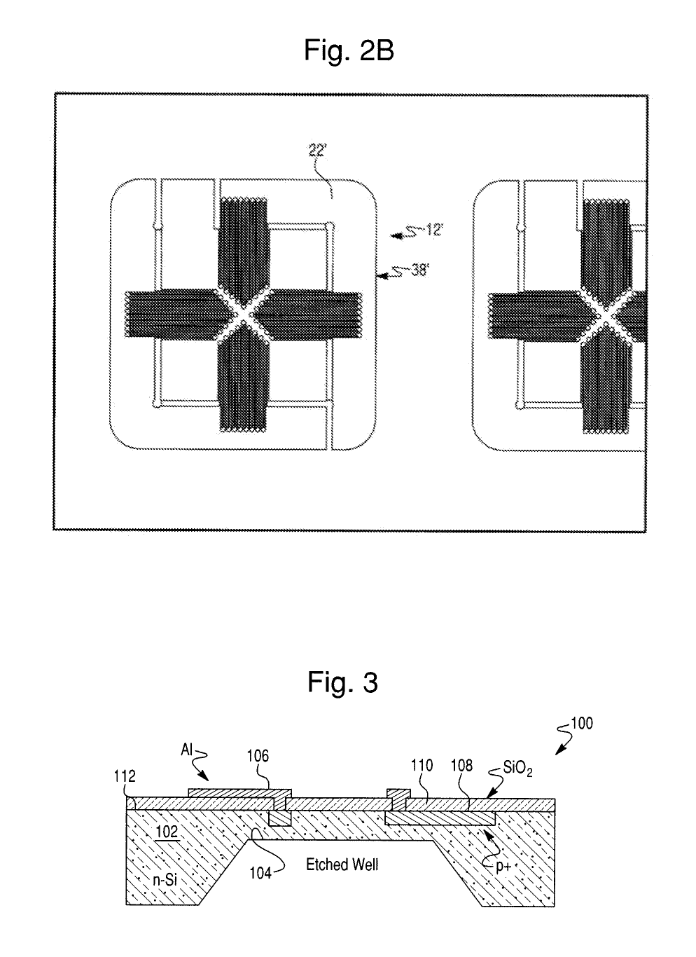

Referring to FIGS. 2A and 2B, an alternative embodiment of the detector is illustrated as detector 10′. In this embodiment, sensing and reference junctions 14′, 16′ are both disposed on membrane 22′ so that the sensing and reference junctions will respond similarly to changes in ambient temperature. Micrographs of sensor 12′ are shown in FIGS. 2A (top view) and 2B (bottom view).

Referring to FIG. 3, another alternative embodiment of a sensor is illustrated as sensor 100. In this embodiment, the plate comprises a semiconductive material, such as silicon, and the thermocouples may be fabricated thereon using silicon (doped with boron) and aluminum as the conductive materials. Sensor 100 comprises a semiconductive plate 102 of n-type material. Sensor 100 comprises an etched well 104 which functions to increase the sensitivity of sensor 100 and speed up the response to detected temperature variations. Thermocouples are fabricated on substrate 102 and comprise a first conductive material 106 (e.g., aluminum) and a second conductive material 108 (e.g., p-type silicon). An insulative layer 110, such as silicon dioxide, is formed over a top surface 112 of plate 102, which insulates the major portions of corresponding first and second conductive materials in the plurality of thermocouples comprising the thermopile. Sensor 100 may measure about 10 mm by 10 mm in the illustrated embodiment, though other sizes are contemplated.

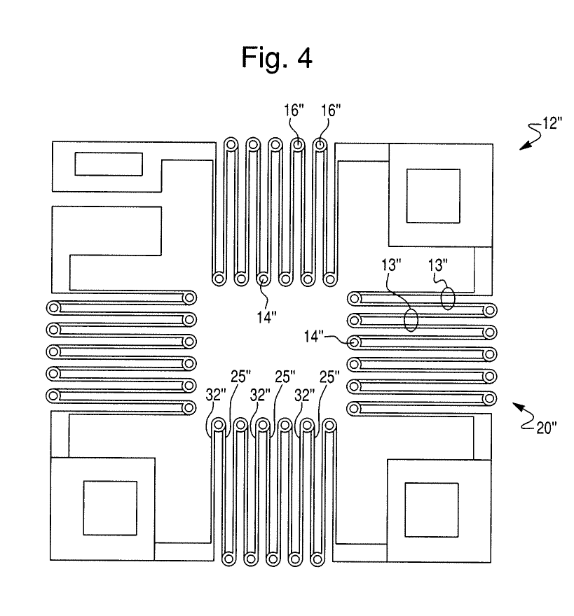

Referring now to FIG. 4, a thermoelectric sensor 12″ is shown according to another exemplary embodiment. In this embodiment, sensor 12″ comprises twenty-four sensing junctions 14″ and twenty-four reference junctions 16″ around a perimeter 20″. Each of thermocouples 13″ comprises two materials, each material having a line width of approximately 80 micrometers. Perimeter 20″ measures approximately 10 mm by 10 mm. Every other strip 25″ comprises a first conductive or semiconductive material and alternative strips 32″ comprise a second conductive or semiconductive material.

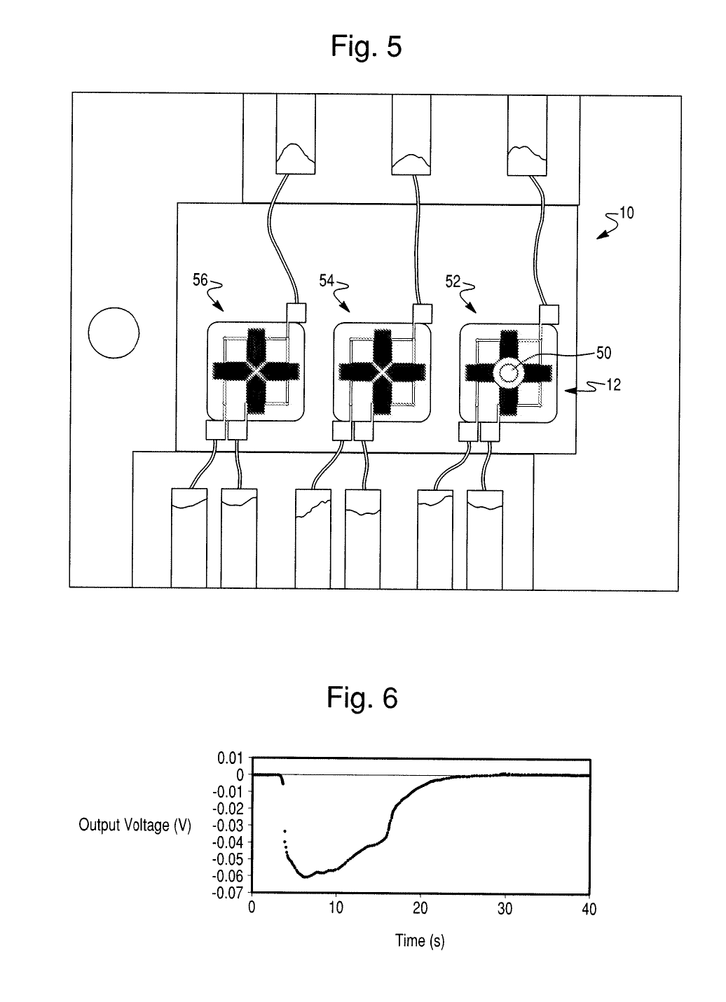

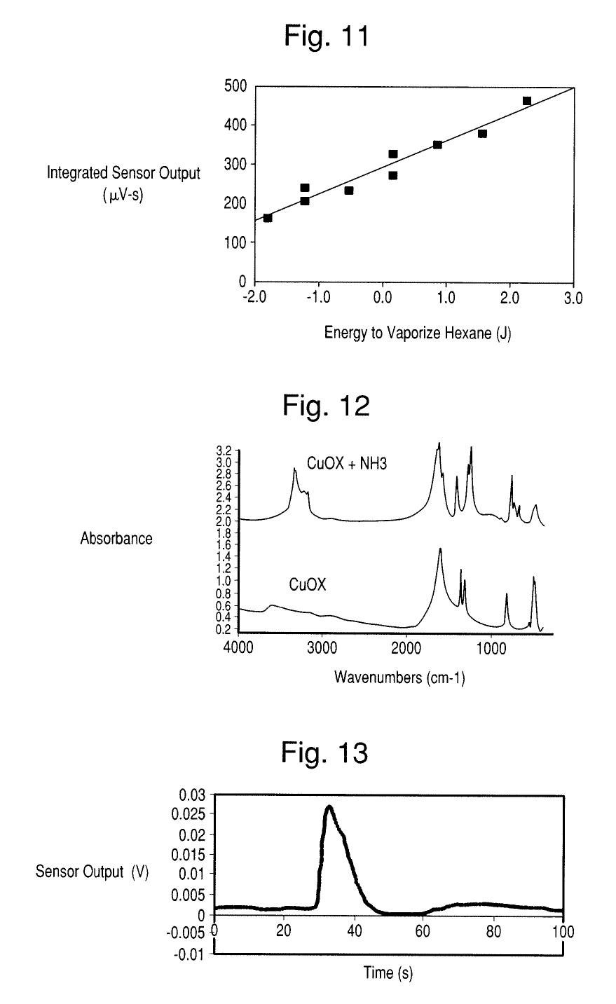

Referring now to FIG. 5, detector 10 is shown comprising thermoelectric sensor 12 (which may be any of the alternative sensor embodiments referenced above or other sensor embodiments) and a reactive layer 50 coupled to sensor 12. Reactive layer 50 is also shown in FIG. 1B. In FIG. 1B, layer 50 is directly over the sensing junctions in order to enhance heat transfer from layer 50 to the sensing junctions. Reactive layer 50 comprises a material (e.g., a chemical or biological material) which is reactive with an airborne chemical or airborne biological agent of interest, the reaction being exothermic or endothermic. When the airborne agent of interest reacts with layer 50, the reaction is detected by thermoelectric sensor 12, which provides an electrical signal which may provide an indication based on the reaction. A temperature change (e.g., heat released or absorbed) on layer 50 as a result of the reaction is detected by sensor 12. The electrical signal output by sensor 12 may be a voltage, current or other signal, and may be indicative of a temperature, a change in temperature, a thermal event, or other indication indicating the presence or quantity of the agent of interest. The electrical signal output by sensor 12 may be integrated. The integrated output signal may be related to the concentration of the agent of interest, as will be described in exemplary form with reference to FIG. 11 below. The relationship may alternatively be linear, curvilinear, such as logarithmic, or have some other relationship.

The agent of interest is not limited in any respect. For example, the agent of interest may be chemical, biological, or other, and may be a noxious pollutant, or other threat to animal, human or public health or safety. The agent of interest may be in vapor or gas-phase, aerosol, liquid, or other form.

As noted above, reactive layer 50 comprises a material (e.g., a chemical and/or biological material) which is reactive with an airborne chemical or airborne biological agent of interest, the reaction being exothermic or endothermic. The material may be any chemical material (including mixtures of two or more materials) reactive in an exothermic or endothermic reaction with an airborne chemical or airborne biological agent of interest, and/or any biological material (including mixtures of two or more materials) reactive in an exothermic or endothermic reaction with an airborne chemical or airborne biological agent. Examples of materials and corresponding agents of interest are exemplified below.

Layer 50 may by applied as a coating to cover and make contact with at least a portion of thermoelectric sensor 12 and, more specifically, to make contact with sensing junctions 14 of sensor 12. Layer 50 and sensor 12 may comprise an integrated device with layer 50 integral to sensor 12. Layer 50 may be of any shape or size, and is shown in a circular shape in the illustrated embodiment, though square, oval, rectangular, oblong shapes, etc. are contemplated.

In some embodiments, layer 50 is selective, i.e., it reacts with one or a limited number of chemical or biological agents of interest, or with one or a limited number of classes of chemical or biological agents of interest. In some embodiments, the reactive material will react with a sufficient selectivity to a baseline substance within the agent of interest, such that the reactive material will discriminate between normal indoor air substances and substances of concern. In some embodiments, the material reacts rapidly with the target agent at ambient temperatures to give a rapid response time, such as within a few seconds or a few minutes.

In some embodiments, layer 50 is compatible with the materials used to fabricate sensor 12. In some embodiments, the material is a solid, or is applied as a solution or suspension that dries to solid or solid form. In some embodiments, the material adheres to the sensor and support (e.g., the polyimide membrane used to support the sensor).

Exemplary layers 50 and corresponding target agents of interest are illustrated in the table below.

| Layer | Agent of Interest |
|---|---|
| Metal compounds (e.g., CuOX) | Basic vapors (e.g., NH₃, pyridine) |
| Metal hydroxide compounds | Acidic vapors (e.g., HCN, HCl) |
| (e.g., Mg(OH)₂) | |
| Ligand-binding proteins (e.g., avidin) | Ligand (e.g., biotin) |
| Antibody or receptor/ligand protein | Antigen or ligand/receptor binding |
| | protein (in solution or as |
| | components of bacterial cells, |
| | spores, or virus particles) |
| Polymeric materials | Volatile organic compounds |

Other agents of interest that may include cyanide, nerve agents such as sarin gas and mustard gases, and industrial chemicals, such as chlorine, sulfuric acid, nitrous acid, and other carcinogenic or organic chemicals, can be detected with appropriate combinations of metal complexes, metal hydroxides, and catalytic oxidation sensors. Pathogenic bacteria, fungi (e.g., sporulating), and viruses that could be aerosolized within a building may also be detected.

In some embodiments, the material is chosen to be reactive in an acid-base chemical reaction with the target agent. The chemistry of acid-base reactions has been investigated in detail and is well-known and well-understood. Acid-base reactions occur rapidly at room temperature, particularly in solution, and often produce large heat changes. In addition, many solid acids and bases are known.

Gaseous acids like HCl or HCN and bases such as NH₃ are environmental hazards and there is considerable interest in sensors for these types of materials. B. Timmer, W. Olthuis, A. van den Berg, “Ammonia Sensors and their application—a review,” Sensors and Actuators B 107, (2007), 666-677.

An exemplary method for testing for chemical reactivity between a solid and vapor molecules comprises placing a plastic weighing boat containing a carefully weighed amount of the solid compound into a capped 2000 ml beaker containing vapor in equilibrium with a solution of the test molecule. Color change, mass change, and infrared (IR) spectroscopy may be used to determine if a reaction has occurred and to estimate the rate of reaction.

A prototype base may be ammonia and a prototype acid may be acetic acid. Salts of nickel, copper, and zinc have been found to react with ammonia vapor and reactions between the oxalate salts of these metals and ammonia were selected for use because these salts are inexpensive, readily available, do not dissolve in water and adhere to the polyimide film.

The rate of heat released (q) during a chemical reaction can be calculated if the rate of reaction and the enthalpy change (ΔH) are known:
q=joules/unit time=reaction rate(mass/time)*ΔH(J/mass).

Where these quantities are not known, the following, procedures to measure them can be used.

The rate of reaction can be measured by determining the mass change that occurs when the coating compound (e.g., reactive material) is placed in the headspace of a covered 2-liter beaker containing 50 ml of ammonia solution for a fixed amount of time (usually one minute). The complete rate expression can be established by repeating the experiment several times with different masses of the coating compound and with different partial pressures of ammonia vapor produced by changing the concentration of the ammonia solutions in the beaker.

The enthalpy change can be determined using differential scanning calorimetry (DSC). The reaction product produced in the beaker experiments is heated in the DSC to decompose the compound. The enthalpy changes observed as the decomposition progressed can be used to estimate ΔH for the ammonia vapor reaction.

Nickel, copper, and zinc oxalate reacted rapidly with ammonia. Since the copper compound does not have waters of hydration, it was anticipated that it would have the largest ΔH. New bands at ˜3300 cm⁻¹, ˜1400 cm⁻¹, and ˜950 cm⁻¹ that can readily be assigned as the N—H stretch, the H—N—H bend and the umbrella modes of the complexed ammonia respectively were observed in the IR spectrum when copper oxalate was exposed to NH₃ vapor. This confirmed that a rapid reaction had occurred. See FIG. 12, in which changes in the IR spectrum were observed when ammonia reacted with copper oxalate.

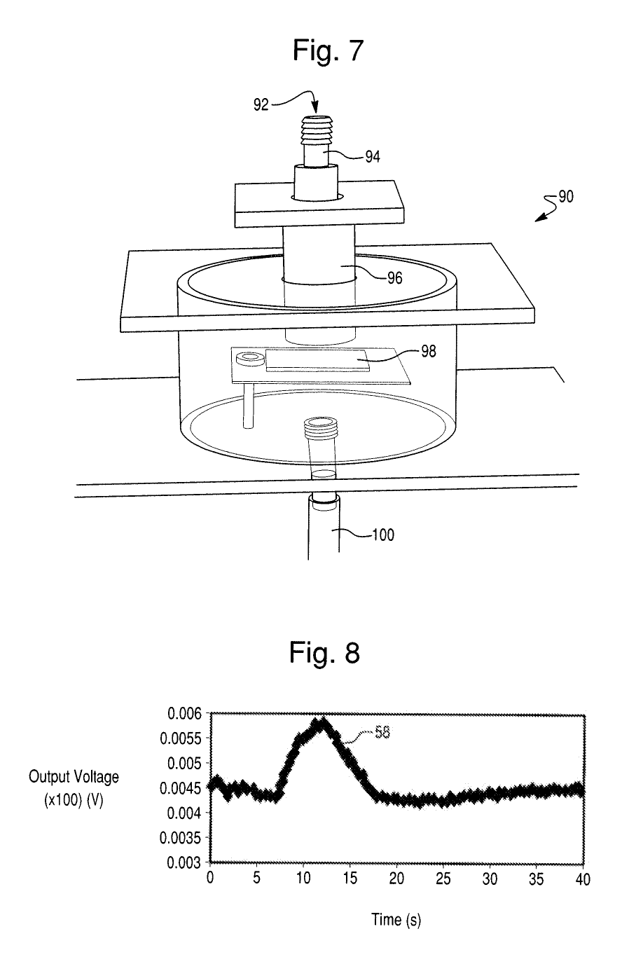

To verify the ability of the sensor to detect target agents, a flow tube apparatus 90 shown in FIG. 7 may be used for exposing the sensors to air streams containing various pulsed concentrations of, for example, ammonia. Air is drawn through an inlet 92 at a top portion 94, passes down a flow tube 96, and floods a surface 98 of the array, shown mounted (sensor leads are not shown in this figure). Air flow is drawn by a sampling pump through a bottom outlet 100. Injections of ammonia vapor into the inlet air stream create pulses of ammonia at known concentrations. As illustrated, the arrays have one sensor coated with a reactive material and two uncoated sensors. Air temperature variability is sensed by all three sensors; the ammonia pulse is detected by the difference in signals from the coated and uncoated sensors. A similar apparatus and procedure may be implemented for testing bioaerosols and other agents.

Heat and mass transfer calculations suggest the feasibility of a thermoelectric sensor suitable for detecting agents of interest. The calculations below indicate that the energy flux to a sensor from an air concentration of 1000 ppm of a gaseous substance would be roughly 2×10⁻⁴ J/s to 5×10⁻⁴ J/s.

Since our thermopiles have sensitivities >4 V-s/J, we can estimate the sensor output voltage resulting from the above values of energy flux by multiplying (energy flux)×(sensitivity)=8×10⁻⁴ V to 20×10⁻⁴ V. If the coated area of the sensor is smaller, say closer to 2 mm×2 mm rather than 10×10, then our expected output is reduced to 3.2×10⁻⁵ V to 8×10⁻⁵ V, but this is still detectable and measurable.

Assumptions of Initial Calculations:

1. The mass flux to the sensor surface would be proportional to the mass diffusivity of the substance in air and the concentration difference between the air concentration in the room and the concentration at the surface of the coating. In addition, the mass flux would be inversely proportional to the thickness of the “film” above the sensor surface (i.e., the distance over which the concentration gradient exists). In simple math terms, F=D·ΔC/Δx.

2. For mass diffusivity in air, a typical value for organic vapors of 0.05 cm²/s (5×10⁻⁶ m²/s) from a handbook (Perry's Chemical Engineers).

3. Air-phase concentration at the surface of the sensor coating of zero, so ΔC=1000 ppm≈0.04 mole/m³.

4. The film thickness, Δx, of 1 cm, or 10⁻² m. Probably not off by more than a couple of orders of magnitude from actual applications.

5. Putting these numbers together gives a mass flux of 2×10⁻⁵ mole/s-m².

6. If the coated area of the sensor is 10 mm×10 mm, or 10⁻⁴ m², the mass rate to an individual sensor would be roughly 2×10⁻⁹ mole/s.

7. Heat of reaction with the coating on the sensor=1000 kcal/mole≈4.2×10⁶ J/mole.

8. Multiplying #6 times #7 gives an energy flow rate to a sensor of roughly 10⁻² J/s.

A more typical heat of reaction would be about 50 kcal/mole (1000 kcal/mole assumption above was based on a typical heat of combustion). Using this value leads to an energy flow rate of roughly 5×10⁻⁴ J/s.

An alternative calculation uses data from the following article, which reports experimentally determined sorption rates, to estimate the mass transfer rate to a sensor: Won, D., R. L. Corsi, and M. Rynes, “Sorptive interactions between VOCs and indoor materials,” Indoor Air, 11, 246-256 (2001).
dM/dt=kₐC_{g}−Mⁿ

where M=mass on sink surface, mg/m², n=“a constant that accounts for nonlinearities in the desorption process,” kₐ=adsorption rate coefficient, m/h, k_{d}=desorption rate coefficient, 1/h (if n=1), and C_{g}32 VOC concentration in air, mg/m³.

In our case, assume irreversible sink, so k_{d}=0. Therefore,
dM/dt=kₐC_{g}, or dm/dt=kₐAC
where m=mass being sorbed, moles, A=adsorption area, m², C=concentration in air, moles/m³.

If kₐ=1 m/h (a high “average” value from Table 2 in the Won et al. article):
A=10⁻⁴m²
C=1000 ppm≅0.04 mole/m³
So dm/dt=(1)(10⁻⁴)(0.04)=4×10⁻⁶ mole/h=1×10⁻⁹ mole/s. Energy flux to the sensor=1×10⁻⁹ mole/s×50 kcal/mole×4.2×10³ J/kcal≅2×10⁻⁴ J/s.
This is within a factor of about 2 of the rate calculated by the first method, so the agreement is good.

Calculations for Particulate Pollutants, from N. A. Fuchs, Mechanics of Aerosols:
Diffusivity of 1 μm particle=1.3×10⁻⁷ cm²/s
For 5 μm particle=2.4×10⁻⁸ cm²/s.

These diffusivities are roughly 2-3 orders of magnitude less than for gases, but the total moles of reacting substance would depend on its concentration on the surface of the particles.

In some embodiments, the biological reactive agent is a member of a binding pair, and the complementary member of the binding pair is associated with the agent of interest. As used herein, “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate, lgG-protein A, antigen-antibody, and the like.

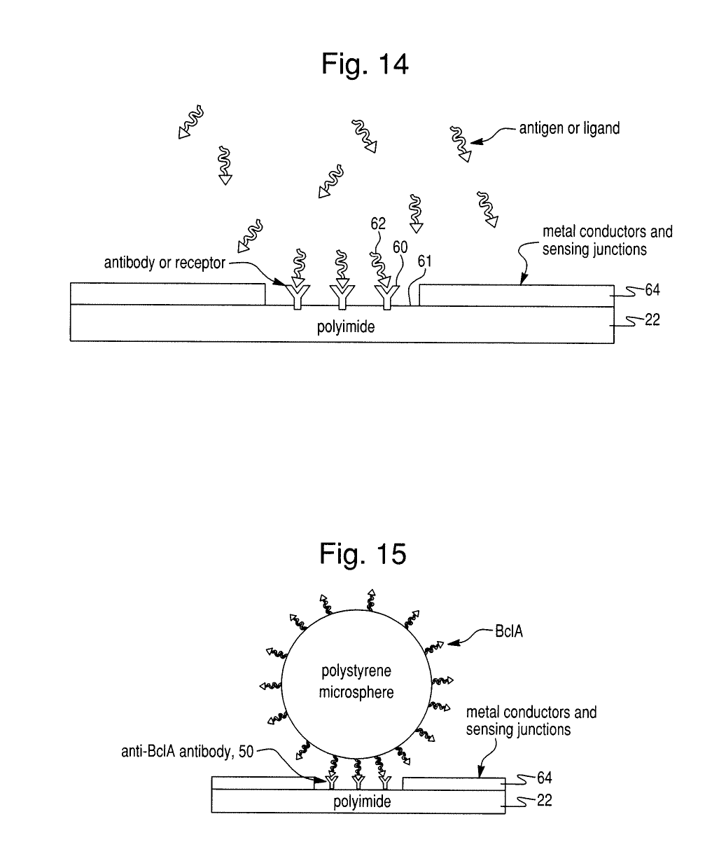

Referring now to FIG. 14, layer 50 may comprise at least one biological reactive agent 60, such as an antibody (e.g., monoclonal, polyclonal, single chain, recombinant, etc.) or receptor or ligand protein reactive with a biological airborne agent of interest 62. Antibodies against antigens associated with a number of biological agents of interest are known, and methods of making antibodies for other antigens are well known. For example, antibodies against viruses, bacteria, pathogenic organisms, infectious agents, toxins and chemicals are known and others can be made using routine methods. Additionally or alternatively, the material may comprise a ligand or receptor protein that binds to its partner receptor or ligand, respectively. Ligand and receptor protein pairs are well-known in the art. Additionally or alternatively, layer 50 may comprise a protein, such as avidin, which can be used when the ligand biotin is used for system characterization. Biotin binding to the protein avidin produces moderate releases of heat energy. Swamy, M. J., “Thermodynamic analysis of biotin binding to avidin—a high-sensitivity titration calorimetric study,” Biochemistery and Molecular Biology International 36(1), (1995), 219-225. Moy, V. T., E. L. Florin, H. E. Gaub, “Intermolecular forces and energies between ligands and receptors,” Science 266, (1994), 257-259.

Biological agents which may be detected include and which are candidates for bioterrorism include bacterial pathogens, such as Yersinia pestis (plague), Burkholderia mallei (glanders), Burkholderia pseudomallei (melioidosis), Francisella tularensis (tularemia), and Bacillus anthracis (anthrax), and viruses, such as Smallpox, Ebola, and Marburg (hemorrhagic fever). See also Linda J. Utrup and Allan H. Frey, “Fate of Bioterrorism-Relevant Viruses and Bacteria, Including Spores, Aerosolized into an Indoor Air Environment Experimental Biology and Medicine,” 229:345-350 (2004) Randomline, Inc., Potomac, Md. 20854 and GAO Report to Congressional Requestors GAO-03-139 Federal Bioterrorism IT, entitled “Information Technology Strategy Could Strengthen Federal Agencies' Abilities to Respond to Public Health Emergencies.” In the Utrup and Frey article, ‘mock’ pathogens were used to determine the nature of the distribution of bacteria and viruses throughout a room with normal ventilation, electrical conduits, etc. These detectors may have use in monitoring for ‘sick building’ conditions, for example, testing for the types of molds that grow on walls, under carpets, etc., i.e., molds that produce airborne spores which lead to allergic reactions in some people.

The biological reactive agent 60 (e.g., antibody, receptor protein, etc.) is disposed on membrane 22. Biological reactive agent 60 may comprise an Fc fragment coupled to a surface 61 of membrane 22 and a Fab fragment coupled to the Fc fragment and configured to bind the antigen associated with the agent of interest. Additionally or alternatively, biological reactive agent 60 may comprise a protein (e.g., one produced by recombinant biotechnology) configured to bind to its partner (“binding protein”) or ligand, respectively.

Conductor/sensing junction layer 64 represents various thermoelectric sensor elements described in greater detail with reference to FIGS. 1-4 above. In the illustrated embodiment, layer 50 is configured to detect bio-aerosols comprising antigen associated with an agent of interest. The antibodies disposed on the detector surface are chosen to specifically recognize and bind the antigen.

As illustrated in FIG. 15, layer 50 comprises an anti-BclA antibody. BclA is an antigen associated with anthrax spores. Conductor/sensing junction layer 64 represents various thermoelectric sensor elements described in greater detail with reference to FIGS. 1-4 above. When the BclA antigen (in the form of a ‘mock’ spore) comes into contact with layer 50, the antibody recognizes and binds the antigen in an exothermic reaction. Thus, this detector may be used to detect bioaerosols, such as anthrax spores. The ‘mock’ spore refers to the microsphere system described in the paragraph below, except that the protein BclA would be bound to the microsphere (instead of biotin).

“Model” systems to demonstrate the detection of biological agents of interest were designed. One system uses commercially available microspheres to which are bound molecules of biotin. Such microspheres can be exposed to an airflow within a chamber containing a thermopile detector to which the protein avidin is bound, as described above. Biotin/avidin binding may be determined by detection of transient temperature increases. Another system uses actual bacterial spores, obtained from non-pathogenic bacteria genetically similar to Bacillus anthracis (anthrax), which may be generated by readily available sporulation culture materials and techniques. These spores, and/or purified outer spore surfaces, may be used to raise antibodies that specifically recognize and bind to the spores. These antibodies may be disposed on a sensor, such as via binding to polyimide membrane to which a thermopile is mounted, as described above. Spores, dried and/or aerosolized, can be introduced into an airflow and spore-antibody binding can be detected by transient temperature increases detected by the thermopile.

### EXAMPLE

An example of an array of three sensors 52, 54 and 56 is shown in FIG. 5. Sensor 52 was fabricated as follows:

1. An aluminum plate 25 mm×38 mm and 1.7 mm thick was machined.

2. Three square holes 9.3 mm×9.3 mm were machined in the plate. For devices as described with reference to FIGS. 1A and 1B, the square holes were smaller (6.0 mm×6.0 mm) so that when completed, the reference junctions were disposed on the membrane material directly over aluminum, so that they are “thermally grounded.”

3. The top and bottom surfaces of the aluminum plate were polished flat and the plate was cleaned.

4. The polyimide (PI) membrane material (25 μm thick) was applied to the top surface of the aluminum plate and attached with a silicone adhesive. Alternatively, a polyethylene terephthalate (PET) membrane material can be used, and an epoxy adhesive can be used rather than silicone adhesive.

5. After cleaning, photoresist was coated on the polyimide and it was photolithographically patterned in preparation for standard “liftoff” processing. This left photoresist on all areas of the polyimide surface where no bismuth was wanted, with open windows in the photoresist where the bismuth was wanted, e.g., where the bismuth metal stripes were to be formed.

6. Bismuth was deposited on the patterned side of the sample by thermal evaporation. The bismuth thickness was approximately 350 nm.

7. The sample was placed in acetone baths which were gently agitated with ultrasonic energy in order to dissolve the photoresist, in the process lifting off all the unwanted bismuth. Bismuth was left behind only in the desired areas, specifically where there were open windows in the photoresist pattern.

8. Photoresist was again coated on the sample surface and it was again photolithographically patterned in preparation for standard “liftoff” processing. This time photoresist was left on all areas of the surface where no antimony was wanted, with open windows in the photoresist where the antimony was wanted, e.g., where the antimony metal stripes were to be formed.

9. Antimony was deposited on the patterned side of the sample by thermal evaporation. The antimony thickness was approximately 350 nm.

10. The sample was placed in acetone baths which were gently agitated with ultrasonic energy in order to dissolve the photoresist, in the process lifting off all the unwanted antimony. Antimony was left behind only in the desired areas, specifically where there were open windows in the photoresist pattern.

11. As was shown in FIGS. 1A and 1B, the patterns are designed so that the antimony is deposited on top of bismuth in all locations where sensing and reference junctions are desired.

12. The bismuth and antimony stripe widths are approximately 60 μm.

Sensor 52 was coated with copper oxalate in a circular pattern having a diameter of approximately 2 mm to impart chemical sensitivity, in this case sensitivity to ammonia. The flow tube test apparatus of FIG. 7 was used. When sensor 52 was exposed to air containing a short pulse of approximately 30 ppm ammonia at time=approximately 2 seconds, the sensor output voltage changed as shown in FIG. 8. FIG. 8 illustrates a measurable, detectable increase in output voltage at point 58. The rise in the response begins about 6 seconds after introduction of the ammonia.

### EXAMPLE

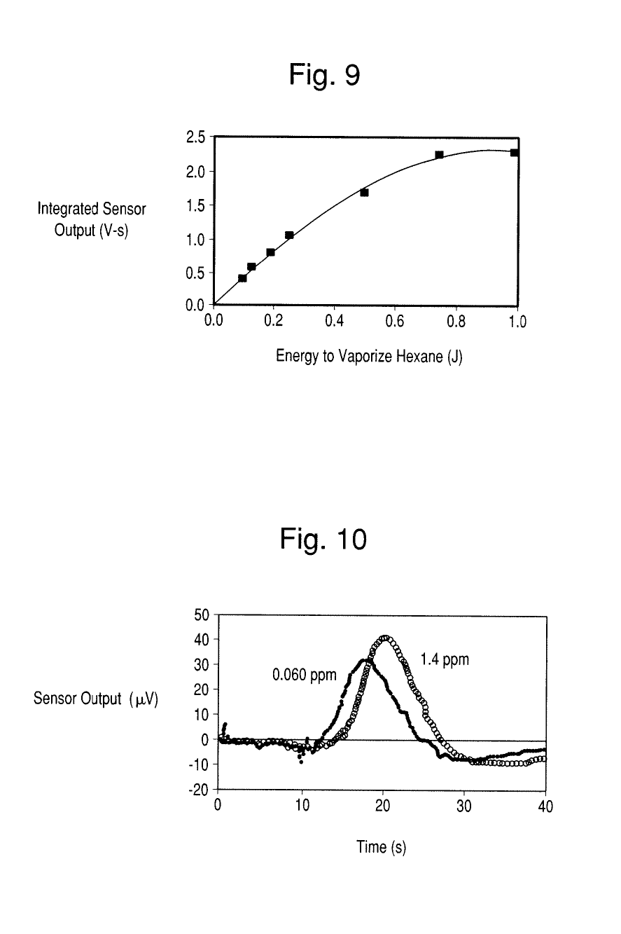

A thermopile was fabricated having a sensitivity of >4 V-s/J, as determined by solvent evaporation and acid-base reactions. This thermopile was fabricated using the same process described above in steps 1-12. In this case, in step 2 the square holes were 6.0 mm×6.0 mm so that when completed, the reference junctions were disposed on the membrane material directly over aluminum, so that they are “thermally grounded”. The device structure is shown in FIGS. 1A and 1B. In order to measure the sensitivity of the thermopiles, measured volumes of a solvent (e.g., hexane) were applied to the sensing junctions and the sensor output voltage was monitored as a function of time. Droplets ranging from 0.4 to 4 micro-liters were used. Graphing the integrals of the voltage-time curves versus the energy required to vaporize the solvent gives the result shown in FIG. 9. The slope for small solvent volumes yields a device sensitivity of >4 V-s/J.

Sensitivity determinations were made from an acid-base reaction. When the sensing junctions are coated with copper oxalate, the device responds to ammonia in the air. Sensors were characterized in a dynamic, flowthrough test chamber (and, in other experiments, in a static apparatus) by measuring their response to short-term exposures to ammonia vapor. Typical sensor responses to approximately 0.060 ppm (injected into the flowthrough test chamber at a concentration of 0.9 ppm) and 1.4 ppm (injected into the flowthrough test chamber at a concentration of 20 ppm) of ammonia vapor are shown in FIG. 10. The 0.9 ppm ammonia pulse was injected at approximately t=7 seconds. The 20 ppm ammonia pulse was injected at approximately t=9 seconds.

These experiments showed sensor responses to ammonia vapor concentrations ranging from <0.1 part per million to 175 parts per million. The integral of these response curves is found to be approximately proportional to the logarithm of the ammonia concentration in the air injected into the test chamber, as shown in FIG. 11. FIG. 11 illustrates several integrated sensor output datapoints and a line approximating a fit to the points. The line is defined by y=68.5x+294, R²=0.954. FIG. 11 also illustrates that sub-ppm concentrations of ammonia can be detected.

### EXAMPLE

A thermopile was fabricated having sensitivities ranging between 3 V/W and 6 V/W. This thermopile was fabricated using the same process as described above with reference to steps 1-12. In this case, in step 2 the square holes were 6.0 mm×6.0 mm so that when completed, the reference junctions were disposed on the membrane material directly over aluminum, so that they are “thermally grounded.” The device structure is shown in FIGS. 1A and 1B.

The output voltage of the thermopile was monitored while applying small (0.5 microLiters to 1.0 microLiters) droplets of hexane to the sensing junctions. Evaporation of the hexane cools the sensing junctions, resulting in a negative output voltage. From the known amount of hexane applied and the heat of vaporization, thermopile sensitivity was determined. The results are illustrated in FIG. 6, which shows an output voltage from a 36-junction thermopile. A 1.0 microLiter drop of hexane was applied to the sensing junctions at t=3.5 seconds. The measured sensitivities ranged from 3 V/W to 6 V/W.

### EXAMPLE

FIG. 13 illustrates sensor output voltage (amplified 100 times) for a thermoelectric sensor having a copper oxalate coating when exposed to ammonia vapor at a concentration of about 70 parts per million. FIG. 13 also shows that the presence of ammonia vapor was detected within about 10 seconds of introducing ammonia to the reactive layer of the sensor.

The success of copper oxalate in detecting ammonia vapors indicates that the Lewis acid-base approach can be used in accordance with the present description to detect gas-phase airborne contaminants. The results indicate that these reactions occur rapidly and that the enthalpy of reaction will limit the sensitivity. Increasing the enthalpy, and thus sensitivity, can be accomplished by a) changing the metal in the coating (e.g., zinc, nickel and cobalt oxalate), and b) modifying the ligand to produce more acidity at the metal, such as using acetate derivates such as trifluoroacetates.

### EXAMPLE

The ability to coat a sensor with a protein was demonstrated as follows. A green fluorescent protein solution was applied to a 50 micrometer thick polyimide membrane in a Petri dish. The solution was dried after 20 minutes at 37 degrees Celsius. The coated membrane was still fluorescent after three weeks, demonstrating the continued presence of undenatured protein.

### EXAMPLE

To determine the ability of a model protein (avidin) to bind to a polyimide surface and retain its ability to bind its ligand biotin: a) A polyimide membrane (mounted on an aluminum support) was plasma etched; 2) Avidin in solution was applied as a droplet for 30 minutes, then excess avidin (unbound) was washed away with deionized water, and the membrane was allowed to dry at room temperature; 3) Biotin (tagged with a FITC (Fluorescein Isothiocyanate) to permit detection of binding by fluorescence) was applied as a droplet for 30 minutes, then excess biotin (unbound) was washed away. Again, the membrane was allowed to dry. Biotin-avidin binding was demonstrated via fluorescence microscopy, showing the ability to bind the protein (in a form that would permit molecular combining with its ligand) to the polymer surface.

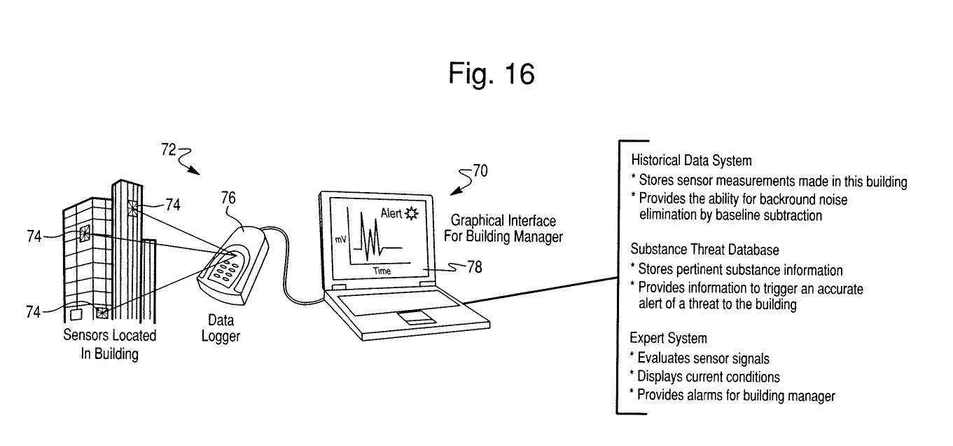

Referring now to FIG. 16, a detection and alarm system 70 may comprise a plurality of detector modules 74 disposed throughout a building (e.g., in a ventilation duct, room, etc.). Each module 74 may comprise a plurality of detectors 10 that detect heat released or absorbed when airborne agents of interest react with specific surfaces. Each of detectors 10 may comprise a reactive layer reactive with the same or different airborne chemical or airborne biological agent of interest. Detectors 10 may comprise at least three detectors, or any number of detectors.

In some embodiments, a control system 72 is coupled to detector modules 74 and is configured to receive the electrical signals, to determine when an alarm condition exists, and/or to provide an alarm output signal based on the determination. For example, control system 72 may be configured to provide the alarm output signal when the electrical signals indicate the presence of an agent of interest, an agent of interest in a concentration exceeding a predetermined level, or a sudden increase in concentration of the agent (e.g., in excess of a predetermine rate of increase). The alarm may be audible, visual, an e-mail, a control signal to control a building system (such as a heating ventilation and air conditioning (HVAC) system, alarm system, evacuation alarm, etc), etc.

In some embodiments, detectors 10 may further be combined with and complement detectors using other sensing mechanisms (e.g., mass, resistance, or optical changes, etc.) in a single module 74 or multiple modules as part of detection and alarm system 70.

In some embodiments, control system 72 may further comprise a data logger 76 and a computer 78, which may be separate components or combined into one computer (e.g., a server and client arrangement, a laptop, desktop, handheld computer, etc.). Computer 78 may be configured to store sensor measurements from detectors 10. Computer 78 may be configured to compare sensor measurements against a database of predetermined data for the agents of interest (e.g., thresholds) and optionally may provide or trigger the alarm output signal based on the comparison (e.g., sensor measurements exceeding a predetermined threshold, sensor measurements indicating a rapid rise in the concentration of the agent of interest, etc.). In some embodiments, computer 78 may further be configured to provide the alarm output signal based at least in part on a baseline level of the agent of interest (e.g., a baseline subtraction) to reduce or eliminate background noise in the data (e.g., trace amounts of the agents of interest, or noise within the system). In some embodiments, computer 78 may further be configured to provide a graphical user interface of current conditions throughout the building, such as, the current levels of agents of interest and whether computer 78 has identified any alert conditions.

In some embodiments, there may be a database within computer 78 and coupled to computer 78 that is configured to store the identity of typical air pollutants in buildings, their physical and chemical properties, and mathematical representations of “normal” variability in air concentrations. In some embodiments, computer 78 may be configured to compare sensor measurements to this database to prevent false positive alarms. In some embodiments, computer 78 may be configured to operate any software, code, or algorithms, such as neural networks, knowledge-based systems, etc.

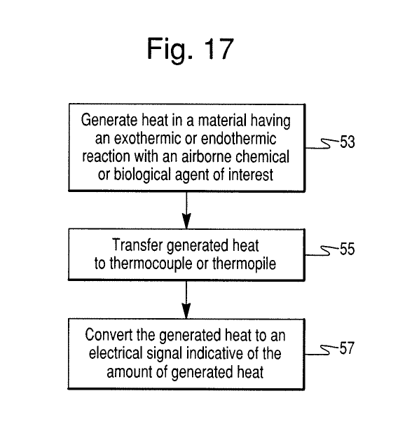

An exemplary method is shown in FIG. 17. At step 53, heat is generated in a material having an exothermic or endothermic reaction with an airborne chemical or biological agent of interest. At step 55, the generated heat is transferred to a thermoelectric sensor, such as a thermocouple or thermopile. At step 57, the generated heat is converted to an electrical signal indicate of the amount of generated heat.

The embodiments illustrated in the FIGS and described above are offered by way of example only. Accordingly, the present invention is not limited to a particular embodiment, but extends to various modifications that nevertheless fall within the scope of the appended claims.

What is claimed is:

1. A detector, comprising:
a thermoelectric sensor configured to sense heat and to provide an electrical signal based on the sensed heat;
a reactive layer coupled to the thermoelectric sensor which is reactive with an airborne chemical or airborne biological agent of interest, the reaction being exothermic or endothermic,
whereby when the airborne agent of interest reacts with the reactive layer, the reaction is detected by the thermoelectric sensor and the electrical signal provides an indication based on the reaction.

2. The detector of claim 1, wherein the thermoelectric sensor comprises a thermocouple.

3. The detector of claim 2, wherein the thermoelectric sensor comprises a thermopile.

4. The detector of claim 1, wherein the reactive layer comprises a chemical compound reactive with a chemical airborne agent of interest.

5. The detector of claim 4, wherein the reactive layer comprises a metal compound.

6. The detector of claim 5, wherein the metal compound is reactive with ammonia.

7. The detector of claim 5, wherein the metal compound comprises copper oxalate.

8. The detector of claim 4, wherein the reactive layer is configured to have an acid-base reaction with the chemical airborne agent of interest.

9. The detector of claim 1, wherein the reactive layer comprises at least one biological reactive agent reactive with a biological airborne agent of interest.

10. The detector of claim 9, wherein the reactive layer comprises an antibody that specifically binds to the biological airborne agent of interest.

11. The detector of claim 9, wherein the protein comprises avidin and the biological airborne agent of interest comprises biotin.

12. The detector of claim 1, wherein the reactive layer comprises a polymeric material.

13. The detector of claim 1, an array of at least three thermoelectric sensors comprising different reactive layers reactive to a plurality of different types of airborne chemical or airborne biological agents.

14. The detector of claim 1, wherein the thermoelectric sensor comprises a membrane supported by a plate, the membrane supporting a plurality of thermocouples comprising reference junctions thermally grounded to the plate and sensing junctions clustered near a center of the plate.

15. The detector of claim 1, further comprising a protective coating coupled to the thermoelectric sensor.

16. A method, comprising:
sensing heat on a material having an exothermic or endothermic reaction with an airborne chemical or airborne biological agent of interest; and
converting the sensed heat to an electrical signal indicative of the amount of heat sensed.

17. The method of claim 16, wherein the reaction is a chemical reaction.

18. The method of claim 16, wherein the material comprises a metal compound.

19. A thermoelectric alarm system, comprising:
a plurality of detectors, each detector comprising:
a thermoelectric sensor configured to sense heat and to provide an electrical signal based on the sensed heat; and
a reactive layer coupled to the thermoelectric sensor which is reactive with an airborne chemical or airborne biological agent of interest, the reaction being exothermic or endothermic,
whereby when the airborne agent of interest reacts with the reactive layer, the reaction is detected by the thermoelectric sensor and the electrical signal provides an indication based on the reaction;
wherein each of the detectors comprises a reactive layer reactive with the same or different airborne chemical or airborne biological agent of interest.

20. The thermoelectric alarm system of claim 19, further comprising a control system configured to receive the electrical signals, to determine when an alarm condition exists, and to provide an alarm output signal based on the determination.

21. The thermoelectric alarm system of claim 20, wherein the control system is configured to provide the alarm output signal when the electrical signals indicate an agent of interest in a concentration exceeding a predetermined level.

22. The thermoelectric alarm system of claim 21, wherein the control system is configured to provide the alarm output signal based at least in part on a baseline level of the agent of interest.

23. The thermoelectric alarm system of claim 19, wherein the plurality of detectors comprise reactive layers reactive with different airborne chemical or airborne biological agents of interest.

24. A method, comprising:
generating heat in a material having an exothermic or endothermic reaction with an airborne chemical or biological agent of interest;
transferring the generated heat to a thermoelectric sensor; and
converting the generated heat to an electrical signal indicative of the amount of generated heat.

25. The method of claim 24, further comprising:
generating heat in a second material having an exothermic or endothermic reaction with an airborne chemical or biological agent of interest different than the airborne chemical or biological agent of interest;
transferring the generated heat from the second material to the thermoelectric sensor; and
converting the generated heat from the second material to a second electrical signal indicative of the amount of generated heat.

26. The method of claim 24, further comprising:
receiving the electrical signal;
determining when an alarm condition exists; and
providing an alarm output signal based on the determination.
